(19) United States
(12) Patent Application Publication
(10) Pub. No.: US 2006/0275170 A1
(43) Pub. Date: Dec. 7, 2006

(54) Method for terminal sterilization of transdermal delivery devices

(76) Inventors: Mahmoud Ameri, Fremont, CA (US); Yuh-Fun Maa, Millbrae, CA (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/446,487

(22) Filed: Jun. 1, 2006

Related U.S. Application Data
(60) Provisional application No. 60/687,636, filed on Jun. 2, 2005.

(51) Int. Cl. A61L 2/08

(52) U.S. Cl. 422/22; 53/425

(57) ABSTRACT

An method and system for providing a terminally sterilized transdermal device adapted to delivery a PTH-based agent. A microprojection member that includes a plurality of stratum comeum-piercing microprojections is coated with PTH-based agent formulation an exposed to sufficient radiation to sterilize the microprojection member while retaining sufficient activity of the PTH-based agent. Preferably, the microprojection member is sealed in packing with an inert atmosphere and reduced moisture. The sterilizing radiation can be gamma radiation or e-beam, preferably delivered in a dose in the range of approximately 5-50 kGy. Also preferably, the irradiation is performed at −78.5-25° C. In preferred embodiments, the radiation is delivered at a rate greater than 3.0 kGy/hr.

### CROSS-REFERENCE TO RELATED APPLICATIONS

This application claims the benefit of U.S. Provisional Application No. 60/687,636 filed Jun. 2, 2005.

### FIELD OF THE PRESENT INVENTION

The present invention relates generally to transdermal agent delivery systems and methods. More particularly, the invention relates to methods for sterilizing a transdermal device adapted to deliver a parathyroid hormone agent.

### BACKGROUND OF THE INVENTION

As is well known in the art, osteoporosis is a bone disorder characterized by progressive bone loss that predisposes an individual to an increased risk of fracture, typically in the hip, spine and wrist. The progressive bone loss, which typically begins between the ages of 30 and 40, is mainly asymptomatic until a bone fracture occurs, leading to a high degree of patient morbidity and mortality. Eighty percent of those affected by osteoporosis are women and, based on recent studies, during the six years following the onset of menopause, women lose one third of their bone mass.

As is also well known in the art, parathyroid hormone (PTH) is a hormone secreted by the parathyroid gland that regulates the metabolism of calcium and phosphate in the body. PTH has stirred great interest in the treatment of osteoporosis for its ability to promote bone formation and, hence, dramatically reduced incidence of fractures. Large-scale clinical trials have shown that PTH effectively and safely reduces the percentage of vertebral and non-vertebral fractures in women with osteoporosis.

PTH-based agents have also stirred interest in the treatment of bone fractures (in both men and women) by virtue of their ability to accelerate bone healing.

To this end, various stabilized formulations of PTH-based agents have been developed that can be reconstituted for subcutaneous injection, which, as discussed below, is the conventional means of delivery. Illustrative are the formulations disclosed in U.S. Pat. No. 5,563,122 (“Stabilized Parathyroid Hormone Composition”) and U.S. Patent Application Pub. No. 2002/0107200 (“Stabilized Teriparatide Solutions”), which are incorporated by reference herein in their entirety.

A currently approved injectable PTH-based agent is FORTEO™ (an rDNA derived teriparatide injection), which contains recombinant human parathyroid hormone (1-34), (rhPTH (1-34)). FORTEO™ is typically prescribed for women with a history of osteoporotic fracture, who have multiple risk factors for fracture, or who have failed or are intolerant of previous osteoporosis therapy, based on a physician's assessment. In postmenopausal women with osteoporosis, FORTEO™ has been found to increase bone mineral density (BMD) and reduce the risk of vertebral and non-vertebral fractures.

FORTEO™ has also been found to increase bone mass in men with primary or hypogonadal osteoporosis who are at high risk for fracture. These include men with a history of osteoporotic fracture, or who have multiple risk factors for fracture, or who have failed or are intolerant to previous osteoporosis therapy. In men with primary or hypogonadal osteoporosis, FORTEO™ has similarly been found to increase BMD.

In addition to subcutaneous injection, other means of delivering PTH-based agents have also been investigated. For example, various pulmonary delivery (i.e., inhalation) methods are discussed in “Pulmonary Delivery of Drugs for Bone Disorders,” Advanced Drug Delivery Reviews, Vol. 42, Issue 3, pp. 239-248 (Aug. 31, 2000), Patton, “Bioavailability of Pulmonary Delivered Peptides and Proteins: -Interferon, Calcitonins and Parathyroid Hormones,” Journal of Controlled Release, Vol. 28, Issues 1-3, pp. 79-85 (January 1994), Patton, et al., “Impact of Formulation and Methods of Pulmonary Delivery on Absorption of Parathyroid Hormone (1-34) from Rat Lungs,” Journal ofpharmaceutical Sciences, Vol. 93, Issue 5, pp. 1241-1252 (May 2004), Codrons, et al., “Systemic Delivery of Parathyroid Hormone (1-34) Using Inhalation Dry Powders in Rats,” Journal of Pharmaceutical Sciences, Vol. 92, Issue 5, pp. 938-950 (May 2003) and Pfützner, A, et al., “Pilot Study with Technosphere/PTH(1-34)—A New Approach for Effective Pulmonary Delivery of Parathyroid Hormone (1-34)”, Horm. Metab. Res., Vol. 35(5), pp. 319-23.

Various methods of active transdermal delivery of PTH-based agents are also discussed in “The Effect of Electroporation on Eontophoretic Eransdermal Delivery of Calcium Regulating Hormones,” Journal of Controlled Release, Vol. 66, Issues 2-3, pp. 127-133 (May 15, 2000) and Chang, et al., “Prevention of Bone Loss in Ovariectomized Rats by Pulsatile Transdermal lontophoretic Administration of Human PTH(1-34),” Journal ofPharmaceutical Sciences, Vol. 91, Issue 2, pp. 350-361 (February 2002).

Despite the efficacy of PTH-based agents in treating disorders such as osteoporosis, there are several drawbacks and disadvantages associated with the disclosed prior art methods of delivering PTH-based agents, particularly, via subcutaneous injection. A major drawback is that subcutaneous injection is a difficult and uncomfortable procedure, which often results in poor patient compliance.

Continuous infusion of PTH-based agent in vivo results in active bone resorption. Based on the efficacy results from the once daily subcutaneous injection, any alternative route of PTH delivery should provide blood concentration of PTH no slower than that for subcutaneously injected PTH. It is therefore of critical importance that the PTH-based agent be administered in a pulsatile fashion.

It would thus be desirable to provide an agent delivery system that facilitates minimally invasive administration of PTH-based agents. It would further be desirable to provide an agent delivery system that provides a pharmacokinetic profile of the PTH-based agent similar to that observed following subcutaneous administration.

Intracutaneous administration of agents, such as hGH, using microprojection systems has previously been documented to provide a pharmacokinetic profile of hGH similar to that observed following subcutaneous administration. See, e.g., Cormier, et al., U.S. Patent Application Pub. No. 2002/0128599, entitled “Transdermal Drug Delivery Devices Having Coated Microprotrusions”.

Transdermal delivery is thus a viable alternative for administering a PTH-based agent that would otherwise need to be delivered via hypodermic injection or intravenous infusion. The word “transdermal”, as used herein, is a generic term that refers to delivery of an active agent (e.g., a therapeutic agent, such as a PTH-based agent or an immunologically active agent, such as a vaccine) through the skin to the local tissue or systemic circulatory system without substantial cutting or penetration of the skin, such as cutting with a surgical knife or piercing the skin with a hypodermic needle. Transdermal agent delivery thus includes intracutaneous, intradermal and intraepidermal delivery via passive diffusion as well as delivery based upon external energy sources, such as electricity (e.g., iontophoresis) and ultrasound (e.g., phonophoresis).

Passive transdermal agent delivery systems, which are more common, typically include a drug reservoir that contains a high concentration of an active agent. The reservoir is adapted to contact the skin, which enables the agent to diffuse through the skin and into the body tissues or bloodstream of a patient.

As is well known in the art, the transdermal drug flux is dependent upon the condition of the skin, the size and physical/chemical properties of the drug molecule, and the concentration gradient across the skin. Because of the low permeability of the skin to many drugs, transdermal delivery has had limited applications. This low permeability is attributed primarily to the stratum corneum, the outermost skin layer which consists of flat, dead cells filled with keratin fibers (i.e., keratinocytes) surrounded by lipid bilayers. This highly-ordered structure of the lipid bilayers confers a relatively impermeable character to the stratum corneum.

One common method of increasing the passive transdermal diffusional agent flux involves mechanically penetrating the outermost skin layer(s) to create micropathways in the skin. There have been many techniques and devices developed to mechanically penetrate or disrupt the outermost skin layers to create pathways into the skin. Illustrative is the drug delivery device disclosed in U.S. Pat. No. 3,964,482.

Other systems and apparatus that employ tiny skin piercing elements to enhance transdermal agent delivery are disclosed in U.S. Pat. Nos. 5,879,326, 3,814,097, 5,250,023, 3,964,482, Reissue No. 25,637, and PCT Publication Nos. WO 96/37155, WO 96/37256, WO 96/17648, WO 97/03718, WO 98/11937, WO 98/00193, WO 97/48440, WO 97/48441, WO 97/48442, WO 98/00193, WO 99/64580, WO 98/28037, WO 98/29298, and WO 98/29365; all incorporated herein by reference in their entirety.

The disclosed systems and apparatus employ piercing elements of various shapes and sizes to pierce the outermost layer (i.e., the stratum comeum) of the skin. The piercing elements disclosed in these references generally extend perpendicularly from a thin, flat member, such as a pad or sheet. The piercing elements in some of these devices are extremely small, some having a microprojection length of only about 25-400 microns and a microprojection thickness of only about 5-50 microns. These tiny piercing/cutting elements make correspondingly small microslits/microcuts in the stratum comeum for enhancing transdermal agent delivery therethrough.

The disclosed systems further typically include a reservoir for holding the agent and also a delivery system to transfer the agent from the reservoir through the stratum comeum, such as by hollow tines of the device itself. One example of such a device is disclosed in WO 93/17754, which has a liquid agent reservoir. The reservoir must, however, be pressurized to force the liquid agent through the tiny tubular elements and into the skin. Disadvantages of such devices include the added complication and expense for adding a pressurizable liquid reservoir and complications due to the presence of a pressure-driven delivery system.

As disclosed in U.S. patent application Ser. No. 10/045,842, which is fully incorporated by reference herein, it is also possible to have the active agent that is to be delivered coated on the microprojections instead of contained in a physical reservoir. This eliminates the necessity of a separate physical reservoir and developing an agent formulation or composition specifically for the reservoir.

As stated, PTH-based agents are at present delivered solely via intravenous routes. It would thus be desirable to provide an agent delivery system that facilitates transdermal administration of a PTH-based agent as well as other parathyroid hormones.

Parenteral pharmaceutical products such as PTH-based agents must meet stringent standards of sterility. One conventional method for assuring a sterile product is aseptic manufacturing. However, the demands of maintaining a sterile environment throughout the manufacturing process are time-consuming, laborious, and extremely expensive.

A potentially attractive alternative to aseptic manufacturing is to sterilize the product at the end of the manufacturing process. Terminal sterilization is used routinely for stable small molecules. Unfortunately, this method presents major challenges for more labile biopharmaceutical products. In particular, complex biological molecular structures such as a PTH-based agent are subject to damage from dislocation of electrons, breakage of covalent bonds, conformational changes, chemical attack from free radicals and oxidation. Thus, such active agents must be protected from degradation to retain therapeutic activity.

In U.S. Pat. Nos. 6,346,216 and 6,171,549, Kent discloses the use of low irradiation rates for the sterilization of various biological molecules. However, these teachings fail to address specific conditions tailored for parathyroid hormones or for transdermal delivery devices. Kent also fails to provide any discussion regarding the effect of packaging on the product's stability and focuses on irradiation at room temperature.

It is therefore an object of the present invention to provide a method for conveniently sterilizing a transdermal device adapted to deliver a PTH-based agent.

It is yet another object of the present invention to provide a method for sterilizing a transdermal delivery system that is more cost efficient than aseptic manufacturing.

Another object of the present invention is to provide a method for terminal sterilization of a PTH-based agent adapted for transdermal delivery.

It is another object of the present invention to provide packaging conditions for a transdermal delivery device that are adapted to optimize stability of a PTH-based agent during sterilization.

Yet another object of the invention is to provide a method for terminally sterilizing a transdermal device for delivering a PTH-based agent so that the agent retains a substantial degree of activity.

### SUMMARY OF THE INVENTION

In accordance with the above objects and those that will be mentioned and will become apparent below, the method and system for terminally sterilizing a transdermal delivery device comprises the steps of providing a microprojection member and exposing the microprojection member to radiation selected from the group consisting of gamma radiation and e-beam, wherein the radiation is sufficient to reach a desired sterility assurance level. The microprojection member includes a plurality of stratum corneum-piercing microprojections with a biocompatible coating having at least one PTH-based agent disposed thereon. Preferably, the microprojection member is sealed within packaging adapted to control environmental conditions surrounding the microprojection member. In one embodiment, the packing comprises a foil pouch.

In one aspect of the invention, sealing a desiccant inside the packaging reduces moisture within the packaging. Alternatively, the microprojection member is mounted on a pre-dried retainer ring prior to sealing the microprojection member inside the packaging. In a preferred embodiment, both a desiccant and a pre-dried retainer ring are used to reduce moisture within the sealed packaging.

In a further embodiment of the invention, the packaging is purged with an inert gas prior to sealing the microprojection member. Preferably, the packaging is purged with dry nitrogen.

The invention also comprises reducing the degradation of the PTH-based agent during sterilization by reducing the temperature at which the irradiation occurs. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

According to the invention, the microprojection member receives a dose of radiation in the range of approximately 5-50 kGy. In one embodiment, the dose is approximately 7 kGy. In another embodiment, the dose is approximately 21 kGy.

In another embodiment, the invention includes exposing the microprojection member to radiation at a rate of greater than approximately 3.0 kGy/hr.

In further embodiments of the invention, the microprojection member is exposed to sufficient radiation to achieve a sterility assurance level of 10⁻⁶.

In other embodiments of the invention, an antioxidant is added to the coating formulation. Suitable antioxidants include methionine and ascorbic acid.

The methods of the invention also comprise sterilizing the microprojection member so that the PTH-based agent retains at least approximately 96% of initial purity. More preferably, the PTH-based agent retains at least approximately 98% of initial purity.

In a currently preferred embodiment of the invention, the method for terminally sterilizing a transdermal delivery device comprises the steps of providing a microprojection member, mounting the microprojection member on a pre-dried retainer ring, sealing the microprojection member inside packaging purged with nitrogen and adapted to control environmental conditions surrounding the microprojection member, and exposing the microprojection member to e-beam radiation, wherein the radiation is sufficient to reach a desired sterility assurance level. The microprojection member preferably includes a plurality of stratum comeum-piercing microprojections having a biocompatible coating formed from a coating formulation having at least one PTH-based agent.

In other embodiments, the method of the invention comprises the steps of providing a microprojection member, placing said microprojection member inside packaging adapted to control environmental conditions, reducing moisture content inside the packaging, sealing said microprojection member with said packaging, and exposing the microprojection member to radiation selected from the group consisting of gamma radiation and e-beam, wherein the radiation is sufficient to reach a desired sterility assurance level. The microprojection member preferably includes a plurality of stratum comeum-piercing microprojections having a biocompatible coating formed from a coating formulation having at least one PTH-based agent.

In additional embodiments, the invention is a transdermal delivery system, comprising a microprojection member including a plurality of microprojections that are adapted to pierce the stratum comeum of a patient having a biocompatible coating disposed on the microprojection member, the coating being formed from a coating formulation having at least one PTH-based agent disposed thereon and packaging purged with inert gas and adapted to control environmental conditions sealed around the microprojection member, wherein the sealed package has been exposed to radiation to sterilize the microprojection member. Preferably, a desiccant is sealed inside the packaging with the microprojection member. Also preferably, the microprojection member is mounted on a pre-dried retainer ring.

In one embodiment of the invention, the packaging is purged with nitrogen.

In another embodiment, the packaging comprises a foil pouch.

In additional embodiments, the invention is a transdermal system adapted to deliver a PTH-based agent, comprising a microprojection member including a plurality of microprojections that are adapted to pierce the stratum comeum of a patient, a hydrogel formulation having at least one PTH-based agent in communication with the microprojection member, and packaging purged with inert gas and adapted to control environmental conditions sealed around the microprojection member, wherein the sealed package has been exposed to radiation to sterilize the microprojection member.

In other embodiments, the invention is a transdermal system adapted to deliver a PTH-based agent, comprising a microprojection member including a plurality of microprojections that are adapted to pierce the stratum corneum of a patient, a solid film having at least one PTH-based agent disposed proximate to the microprojection member, and packaging purged with inert gas and adapted to control environmental conditions sealed around the microprojection member, wherein the sealed package has been exposed to radiation to sterilize the microprojection member. Preferably, the solid film is made by casting a liquid formulation comprising at least one PTH-based agent, a polymeric material, a plasticizing agent, a surfactant and a volatile solvent.

In one embodiment of the invention, the microprojection member has a microprojection density of at least approximately 10 microprojections/cm², more preferably, in the range of at least approximately 200-2000 microprojections/cm².

In one embodiment, the microprojection member is constructed out of stainless steel, titanium, nickel titanium alloys, or similar biocompatible materials.

In another embodiment, the microprojection member is constructed out of a non-conductive material, such as polymeric materials.

Alternatively, the microprojection member can be coated with a non-conductive material, such as Parylene®, or a hydrophobic material, such as Teflon®, silicon or other low energy material.

The coating formulations applied to the microprojection member to form solid biocompatible coatings can comprise aqueous and non-aqueous formulations. In at least one embodiment of the invention, the formulation(s) includes at least one PTH-based agent, which can be dissolved within a biocompatible carrier or suspended within the carrier.

In a preferred embodiment, the PTH-based agent is selected from the group consisting of HPTH(1-34), hPTH salts and analogs, teriparatide and related peptides. Throughout this application, the terms “PTH-based agent” and “HPTH(1-34) agent” include, without limitation, recombinant hPTH(1-34), synthetic hpTH(1-34), PTH(1-34), teriparatide, hPTH(1-34) salts, simple derivatives of hPTH(1-34), such as hPTH(1-34) amide, and closely related molecules, such as hPTH(1-33) or hPTH(1-31) amide, or any other closely related osteogenic peptide. Synthetic hPTH(1-34) is the most preferred PTH agent.

Examples of pharmaceutically acceptable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

In one embodiment of the invention, the PTH comprises in the range of approximately 1-30 wt. % of the coating formulation.

Preferably, the amount of the PTH contained in the coating formulation is in the range of approximately 1-1000 μg, even more preferably, in the range of approximately 10-100 μg.

Also preferably, the transdermally delivered PTH-based agent comprises teriparatide (hPTH (1-34)) and the biocompatible coating comprises a dose of the PTH-based agent in the range of approximately 10-100 μg dose, wherein delivery of the PTH-based agent results in a plasma Cₘₐₓ of at least 50 pg/mL after one application.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a perspective view of a portion of one example of a microprojection member;

FIG. 2 is a perspective view of the microprojection member shown in FIG. 1 having a coating deposited on the microprojections, according to the invention;

FIG. 3 is a side sectional view of a retainer having a microprojection member disposed therein, according to the invention;

FIG. 4 is a perspective view of the retainer shown in FIG. 3;

FIG. 5 is a graph illustrating purity of PTH at varying gamma irradiation and e-beam levels and temperatures, according to the invention;

FIG. 6 is a graph illustrating aggregation of PTH at varying gamma irradiation and e-beam levels and temperatures, according to the invention;

FIG. 7 is a graph illustrating purity of gamma irradiated PTH under selected environmental conditions, according to the invention;

FIG. 8 is a graph illustrating oxidation of gamma irradiated PTH under selected environmental conditions, according to the invention;

FIG. 9 is a graph illustrating the effect of temperature on the purity of gamma irradiated PTH, according to the invention;

FIG. 10 is a graph illustrating the effect of packaging on the purity of gamma irradiated PTH, according to the invention;

FIG. 11 is a graph illustrating the purity of PTH gamma irradiated under selected environmental conditions at varying temperatures, according to the invention;

FIG. 12 is a graph illustrating the purity of PTH gamma irradiated under specific environmental conditions at varying temperatures and irradiation levels, according to the invention;

FIG. 13 is a graph illustrating the purity of PTH at varying gamma irradiation and e-beam levels at varying temperatures, according to the invention;

FIG. 14 is a graph illustrating the percentage change relative to a control for the samples illustrated in FIG. 13, according to the invention; and

FIG. 15 is a graph illustrating the effect of formulation composition on the purity of PTH at varying gamma irradiation and e-beam levels at varying temperatures, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise. Thus, for example, reference to “a peptide” includes two or more such peptides; reference to “a microprojection” includes two or more such microprojections and the like.

### Definitions

The term “transdermal”, as used herein, means the delivery of an agent into and/or through the skin for local or systemic therapy. The term “transdermal” thus means and includes intracutaneous, intradermal and intraepidermal delivery of an agent, such as a peptide, into and/or through the skin via passive diffusion as well as energy-based diffusional delivery, such as iontophoresis and phonophoresis.

The term “transdermal flux”, as used herein, means the rate of transdermal delivery.

The term “co-delivering”, as used herein, means that a supplemental agent(s) is administered transdermally either before the PTH-based agent is delivered, before and during transdermal flux of the PTH-based agent, during transdermal flux of the PTH-based agent, during and after transdermal flux of the PTH-based agent, and/or after transdermal flux of the PTH-based agent. Additionally, two or more PTH-based agents may be formulated in the coatings and/or formulations, resulting in co-delivery of the PTH-based agents.

The terms “PTH-based agent” and “hPTH(1-34) agent”, as used herein, include, without limitation, hPTH(1-34), hPTH salts, HPTH analogs, teriparatide, closely related peptides and agents having a peptide sequence that fimctions by the same means as the 34 N-terminal amino acids (the biologically active region) sequence of the 84-amino acid human parathyroid hormone. The terms “PTH-based agent” and “hPTH(1-34) agent” thus include, without limitation, recombinant hPTH(1-34), synthetic hPTH(1-34), PTH(1-34), hPTH(1-34) salts, teriparatide, simple derivatives of hPTH(1-34), such as HPTH(1-34) amide and closely related molecules, such as HPTHI(1-33) or hPTH(1-31) amide and closely related osteogenic peptides.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

The noted PTH-based agents can also be in various forms, such as free bases, acids, charged or uncharged molecules, components of molecular complexes or nonirritating, pharmacologically acceptable salts.

It is to be understood that more than one PTH-based agent can be incorporated into the agent source, formulations, and/or coatings and/or solid films of this invention, and that the use of the term “PTH-based agent” in no way excludes the use of two or more such peptides.

The term “microprojections” or “microprotrusions”, as used herein, refers to piercing elements which are adapted to pierce or cut through the stratum comeum into the underlying epidermis layer, or epidermis and dermis layers, of the skin of a living animal, particularly, a mammal and, more particularly, a human.

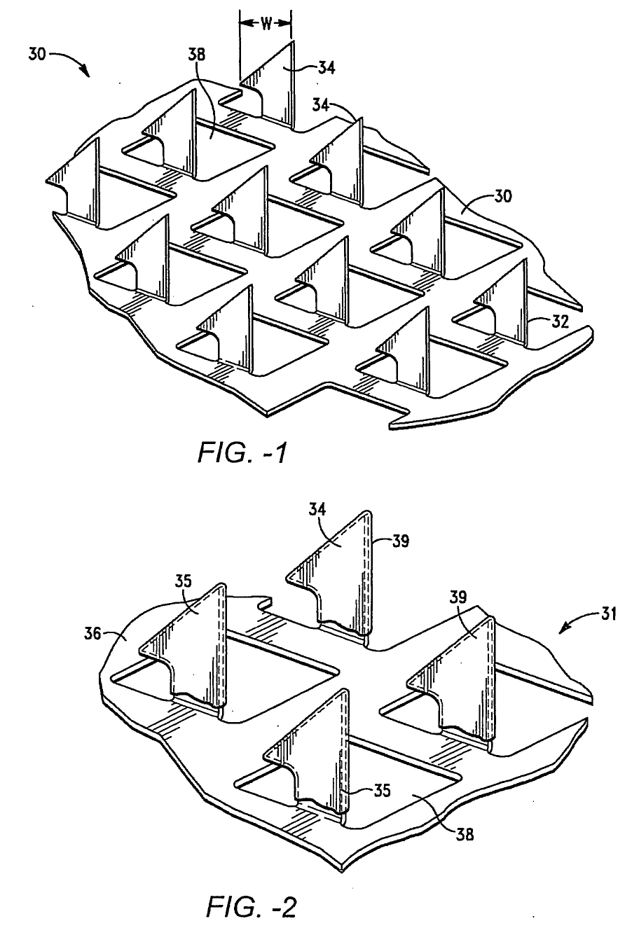

In one embodiment of the invention, the piercing elements have a projection length less than 1000 microns. In a further embodiment, the piercing elements have a projection length of less than 500 microns, more preferably, less than 250 microns. The microprojections further have a width (designated “W” in FIG. 1) in the range of approximately 25-500 microns and a thickness in the range of approximately 10-100 microns. The microprojections may be formed in different shapes, such as needles, blades, pins, punches, and combinations thereof.

The term “microprojection member”, as used herein, generally connotes a microprojection array comprising a plurality of microprojections arranged in an array for piercing the stratum corneum. The microprojection member can be formed by etching or punching a plurality of microprojections from a thin sheet and folding or bending the microprojections out of the plane of the sheet to form a configuration, such as that shown in FIG. 1. The microprojection member can also be formed in other known manners, such as by forming one or more strips having microprojections along an edge of each of the strip(s) as disclosed in U.S. Pat. No. 6,050,988, which is hereby incorporated by reference in its entirety.

The term “coating formulation”, as used herein, is meant to mean and include a freely flowing composition or mixture that is employed to coat the microprojections and/or arrays thereof. The PTH-based agent, if disposed therein, can be in solution or suspension in the formulation.

The term “biocompatible coating” and “solid coating”, as used herein, is meant to mean and include a “coating formulation” in a substantially solid state.

As indicated above, the present invention generally comprises a method for sterilizing a transdermal delivery system at the end of the manufacturing process. The invention also comprises the sterilized delivery systems. The transdermal delivery system includes a microprojection member (or system) having a plurality of microprojections (or array thereof) that are adapted to pierce through the stratum comeum into the underlying epidermis layer, or epidermis and dermis layers. The microprojection member (or system) also includes at least one source or delivery medium of a PTH-based agent (i.e., biocompatible coating, hydrogel formulation, or solid film). The transdermal delivery system is terminally sterilized by exposure to sufficient radiation to achieve a desired sterility assurance level.

Gamma radiation can be delivered by conventional methods, such as by using Cobalt-60 as a radiation source. As one having skill in the art will recognize, a commercial Cobalt-60 sterilizer yields a rate of irradiation in the range of approximately 0.3 Gy/hr and 9.6 kGy/hr. Americium-241 can also be used, and generally irradiate at a rate of approximately 0.3 mGy/hr. Other isotopes can also be used to deliver gamma radiation at a desired rate. E-beam radiation is conventionally generated at substantially higher rates than gamma radiation, such as approximately 100 kGy/hr. In preferred embodiments, the dose rate is 3.0 kGy/hr or greater to minimize the processing time required to achieve a dose sufficient to reach the desired level of sterility.

The radiation dose required for terminal sterilization can be determined by conventional methods. For example, the dose requirements to achieve a sterility assurance level (SAL) of 10⁻⁶ can be assessed from microbiological and manufacturing considerations. In one embodiment, a low dose is based on zero bioburden (8.2kGy using ISO 11137 Method 2B) plus one augmentation (15 kGy) for a sterility failure during the quarterly dose audit. By adding a process capability of +10%, these calculations yield a dose of 16.5 kGy.

Thus, terminal sterilization of the microprojection member loaded with a PTH-based agent is achieved by irradiating the system with e-beam or gamma irradiation. Suitable doses are in the range of approximately 5 to 50 kGy, more preferably, in the range of approximately 10 to 40 kGy. In some embodiments, the dose is at least approximately 7 kGy. In other embodiments, the dose is approximately 14 kGy. In yet other embodiments, the dose is approximately 21 kGy.

In further embodiments of the invention, the microprojection member is mounted on a retainer ring for use with an applicator.

The system can also include packaging adapted to facilitate terminal sterilization of the microprojection member.

In the noted embodiments, it is preferable to maintain an inert, low moisture atmosphere around the microprojection member. Accordingly, the package containing the microprojection member is preferably purged with an inert gas, such as nitrogen or argon. In alternative embodiments, the package can be evacuated to help minimize degradation of the PTH-based agent. In a ftuther embodiment, the amount of oxygen in the packaging is reduced to minimize oxidative degradation.

Further, it is preferable to include a moisture absorbing desiccant in the package. In a currently preferred embodiment, the desiccant comprises a Minipax molecular sieve with 3-4 Å pore size. Other exemplary moisture absorbing materials include, but are not limited to, alumina, bauxite, anhydrous, calcium sulfate, water-absorbing clay, activated bentonite clay, silica gel, or other like materials. The desiccant optionally includes a moisture sensitive color indicator such as cobalt chloride to indicate when the desiccant is no longer operable. The desiccant should be present in an amount sufficient to adsorb any residual moisture from the plastic components of the microprojection system. For example, an amount in the range of approximately 0.5g to 5g of molecular sieve desiccant is sufficient for a typical microprojection system.

In another embodiment, the retainer ring is dried prior to assembly to prevent moisture from the ring being introduced into the sealed packaging.

Yet other embodiments of the invention include an antioxidant to help stabilize the PTH-based agent during irradiation. Suitable antioxidants comprise methionine, ascorbic acid and the like. Preferably, the antioxidant is added in an amount in the range of approximately 1-5%.

In further embodiments of the invention, irradiation of the microprojection member is conducted at reduced temperatures to stabilize the PTH-based agent. In one embodiment, the microprojection member is irradiated at a temperature in the range of approximately −78.5 to 25° C. The microprojection members can be irradiated at a temperature of −78.5° C. under dry ice conditions. In another embodiment, the microprojection member is irradiated at a temperature in the range of approximately 0-25° C. In another embodiment, the microprojection member is irradiated at an ambient temperature in the range of approximately 20-25° C.

Additional information regarding the terminal sterilization of other biologically active agents can be found in co-pending U.S. application Ser. Nos. 60/687,635, filed Jun. 2, 2005, and 60/687,519, filed Jun. 2, 2005, which are hereby incorporated by reference in their entirety.

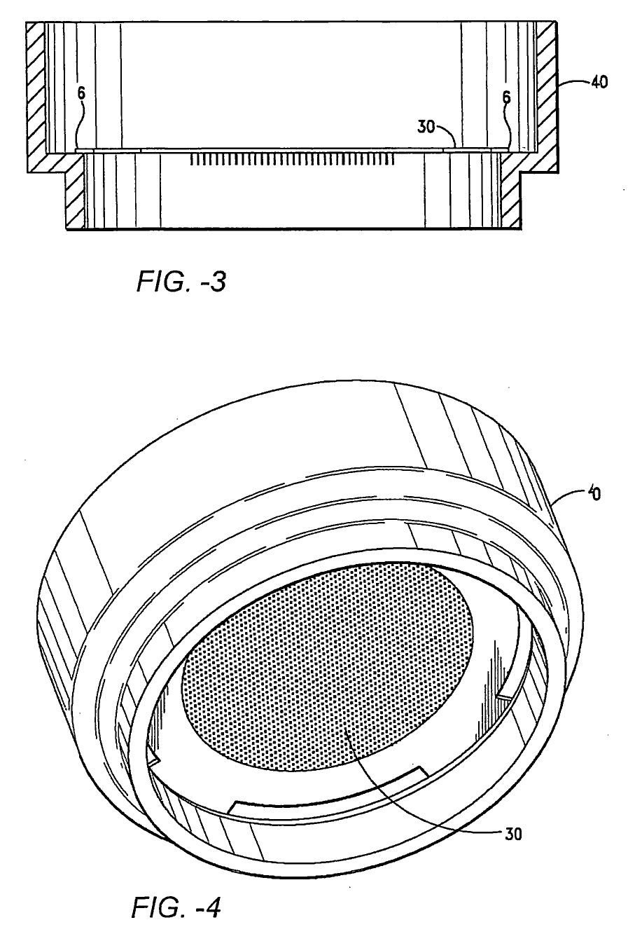

Referring now to FIGS. 1 and 2, there is shown one embodiment of a microprojection member 30 for use with the present invention. As illustrated in FIG. 1, the microprojection member 30 includes a microprojection array 32 having a plurality of microprojections 34. The microprojections 34 preferably extend at substantially a 90° angle from the sheet, which in the noted embodiment includes openings 38. In this embodiment, the microprojections 34 are formed by etching or punching a plurality of microprojections 34 from a thin metal sheet 36 and bending the microprojections 34 out of the plane of the sheet 36.

In one embodiment of the invention, the microprojection member 30 has a microproj ection density of at least approximately 10 microprojections/cm², more preferably, in the range of at least approximately 200-2000 microprojections/cm². Preferably, the number of openings per unit area through which the agent passes is at least approximately 10 openings/cm² and less than about 2000 openings/cm².

As indicated, the microprojections 34 preferably have a projection length less than 1000 microns. In one embodiment, the microprojections 34 have a projection length of less than 500 microns, more preferably, less than 250 microns. The microprojections 34 also preferably have a width in the range of approximately 25-500 microns and thickness in the range of approximately 10-100 microns.

To enhance the biocompatibility of the microprojection member 30 (e.g., to minimize bleeding and irritation following application to the skin of a subject), in a further embodiment, the microprojections 34 preferably have a length less than 145 μm, more preferably, in the range of approximately 50-145 μm, even more preferably, in the range of approximately 70-140 μm. Further, the microprojection member 30 comprises an array preferably having a microprojection density greater than 100 microprojections/cm², more preferably, in the range of approximately 200-3000 microprojections/cm².

The microprojection member 30 can be manufactured from various metals, such as stainless steel, titanium, nickel titanium alloys, or similar biocompatible materials.

According to the invention, the microprojection member 30 can also be constructed out of a non-conductive material, such as a polymer.

Alternatively, the microprojection member can be coated with a non-conductive material, such as Parylene®, or a hydrophobic material, such as Teflon®, silicon or other low energy material. The noted hydrophobic materials and associated base (e.g., photoreist) layers are set forth in U.S. application Ser. No. 60/484,142, which is incorporated by reference herein.

Microprojection members that can be employed with the present invention include, but are not limited to, the members disclosed in U.S. Pat. Nos. 6,083,196, 6,050,988 and 6,091,975, which are incorporated by reference herein in their entirety.

Other microprojection members that can be employed with the present invention include members formed by etching silicon using silicon chip etching techniques or by molding plastic using etched micro-molds, such as the members disclosed U.S. Pat. No. 5,879,326, which is incorporated by reference herein in its entirety.

According to the invention, the PTH-based agent to be administered to a host can be contained in a biocompatible coating that is disposed on the microprojection member 30 or contained in a hydrogel formulation or contained in both the biocompatible coating and the hydrogel formulation. Preferably, the hydrogel formulations of the invention comprise water-based hydrogels. Hydrogels are preferred formulations because of their high water content and biocompatibility. Also preferably, the hydrogel is configured as a gel pack.

In a further embodiment, wherein the microprojection member includes an agent-containing solid film, the PTH-based agent can be contained in the biocompatible coating, hydrogel formulation or solid film, or in all three delivery mediums. Preferably, the solid film is formed by casting a liquid formulation comprising at least one PTH-based agent, a polymeric material, a plasticizing agent, a surfactant and a volatile solvent

In one embodiment, the microprojection member includes a biocompatible coating that contains at least one PTH, preferably, hBNP(1-32). The microprojection member is terminally sterilized to a desired sterility assurance level. Upon piercing the stratum comeum layer of the skin, the peptide-containing coating is dissolved by body fluid (intracellular fluids and extracellular fluids such as interstitial fluid) and released into the skin (i.e., bolus delivery) for systemic therapy. Preferably, a 20 μg bolus dose of a PTH-based agent is delivered in a pulsatile fashion by leaving the microprojection member in place for 15 minutes or less.

Referring now to FIG. 2, there is shown a microprojection member 31 having microprojections 34 that include a biocompatible coating 35 of the PTH-based agent. According to the invention, the coating 35 can partially or completely cover each microprojection 34. For example, the coating 35 can be in a dry pattern coating on the microprojections 34. The coating 35 can also be applied before or after the microprojections 34 are formed. Additional information regarding the use of a transdermal PTH delivery system can be found in co-pending U.S. application Ser. No. 11/084,634, which is hereby incorporated by reference in its entirety.

According to the invention, the coating 35 can be applied to the microprojections 34 by a variety of known methods. Preferably, the coating is only applied to those portions the microprojection member 31 or microprojections 34 that pierce the skin (e.g., tips 39).

One such coating method comprises dip-coating. Dip-coating can be described as a means to coat the microprojections by partially or totally immersing the microprojections 34 into a coating solution. By use of a partial immersion technique, it is possible to limit the coating 35 to only the tips 39 of the microprojections 34.

A further coating method comprises roller coating, which employs a roller coating mechanism that similarly limits the coating 35 to the tips 39 of the microprojections 34. The roller coating method is disclosed in U.S. application Ser. No. 10/099,604 (Pub. No. 2002/0132054), which is incorporated by reference herein in its entirety. As discussed in detail in the noted application, the disclosed roller coating method provides a smooth coating that is not easily dislodged from the microprojections 34 during skin piercing.

According to the invention, the microprojections 34 can further include means adapted to receive and/or enhance the volume of the coating 35, such as apertures (not shown), grooves (not shown), surface irregularities (not shown) or similar modifications, wherein the means provides increased surface area upon which a greater amount of coating can be deposited.

A further coating method that can be employed within the scope of the present invention comprises spray coating. According to the invention, spray coating can encompass formation of an aerosol suspension of the coating composition. In one embodiment, an aerosol suspension having a droplet size of about 10 to 200 picoliters is sprayed onto the microprojections 34 and then dried.

Pattern coating can also be employed to coat the microprojections 34. The pattern coating can be applied using a dispensing system for positioning the deposited liquid onto the microprojection surface. The quantity of the deposited liquid is preferably in the range of 0.1 to 20 nanoliters/microprojection. Examples of suitable precision-metered liquid dispensers are disclosed in U.S. Pat. Nos. 5,916,524; 5,743,960; 5,741,554; and 5,738,728; which are fully incorporated by reference herein.

Microprojection coating formulations or solutions can also be applied using ink jet technology using known solenoid valve dispensers, optional fluid motive means and positioning means which is generally controlled by use of an electric field. Other liquid dispensing technology from the printing industry or similar liquid dispensing technology known in the art can be used for applying the pattern coating of this invention.

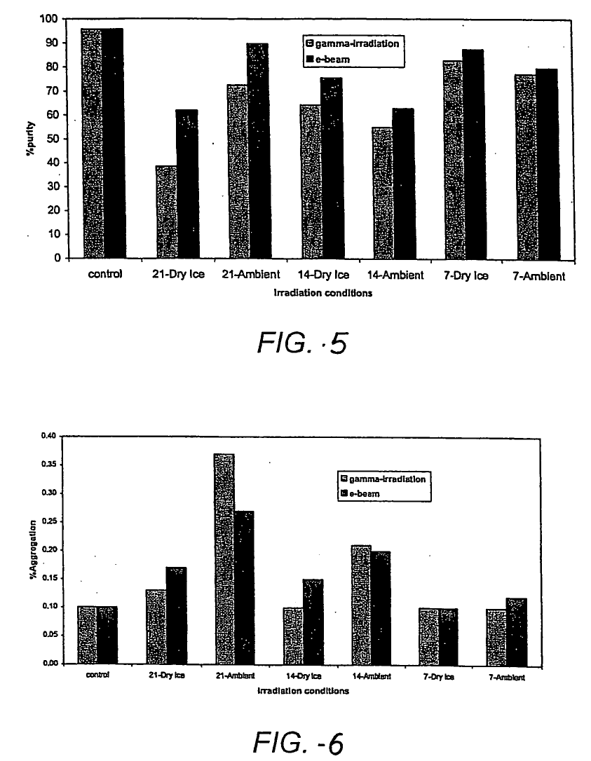

Referring now to FIGS. 3 and 4, for storage and application, the microprojection member 30 is preferably suspended in a retainer ring 40 by adhesive tabs 6, as described in detail in U.S. application Ser. No. 09/976,762 (Pub. No. 2002/0091357), which is incorporated by reference herein in its entirety.

After placement of the microprojection member in the retainer ring 40, the microprojection member is applied to the patient's skin. Preferably, the microprojection member is applied to the patient's skin using an impact applicator, as described in Co-Pending U.S. application Ser. No. 09/976,978, which is incorporated by reference herein in its entirety. As discussed above, retainer ring 40 is preferably pre-dried prior to packaging to reduce the amount of moisture in the atmosphere surrounding the microprojection member during irradiation.

As indicated, according to one embodiment ofthe invention, the coating formulations applied to the microprojection member 30 to form solid biocompatible coatings can comprise aqueous and non-aqueous formulations having at least one PTH-based agent. According to the invention, the PTH-based agent can be dissolved within a biocompatible carrier or suspended within the carrier.

In a preferred embodiment, the PTH-based agent is selected from the group consisting of hPTH(1-34), hPTH salts and analogs, teriparatide and related peptides, including, recombinant hPTH(1-34), synthetic hPTH(1-34), PTH(1-34), teriparatide, hPTH(1-34) salts, simple derivatives of hPTH(1-34), such as hPTH(1-34) amide, and closely related molecules, such as hPTH(1-33) or hPTH(1-31) amide, and any other closely related osteogenic peptide. Synthetic hPTH(1-34) is the most preferred PTH-based agent.

Examples of suitable hPTH salts include, without limitation, acetate, propionate, butyrate, pentanoate, hexanoate, heptanoate, levulinate, chloride, bromide, citrate, succinate, maleate, glycolate, gluconate, glucuronate, 3-hydroxyisobutyrate, tricarballylicate, malonate, adipate, citraconate, glutarate, itaconate, mesaconate, citramalate, dimethylolpropinate, tiglicate, glycerate, methacrylate, isocrotonate, β-hydroxibutyrate, crotonate, angelate, hydracrylate, ascorbate, aspartate, glutamate, 2-hydroxyisobutyrate, lactate, malate, pyruvate, fumarate, tartarate, nitrate, phosphate, benzene, sulfonate, methane sulfonate, sulfate and sulfonate.

In a preferred embodiment, the coating formulation comprises a 1:1 formulation of sucrose:hPTH. Other suitable adjuvants include { human albumin, bioengineered human albumin, polyglutamic acid, polyaspartic acid, polyhistidine, pentosan polysulfate, polyamino acids, sucrose, trehalose, melezitose, raffinose, stachyose, dextran, soluble starch, dextrin, mannitol and inulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactulose, maltose, melibiose, sophorose, and turanose and the like.

The amount and type of adjuvant is adapted to optimize the stability of the PTH during sterilization human albumin, bioengineered human albumin, polyglutamic acid, polyaspartic acid, polyhistidine, pentosan polysulfate, polyamino acids, sucrose, trehalose, melezitose, raffinose, stachyose, dextran, soluble starch, dextrin, mannitol and inulin. Suitable reducing sugars for use in the methods and compositions of the invention include, for example, monosaccharides such as, for example, apiose, arabinose, lyxose, ribose, xylose, digitoxose, fucose, quercitol, quinovose, rhamnose, allose, altrose, fructose, galactose, glucose, gulose, hamamelose, idose, mannose, tagatose, and the like; and disaccharides such as, for example, primeverose, vicianose, rutinose, scillabiose, cellobiose, gentiobiose, lactulose, maltose, melibiose, sophorose, and turanose and the like.

In one embodiment the ratio of adjuvant or a mixture of adjuvants to hPTH(1-34) is between 20:1 to 0.25:1. In a preferred embodiment the ratio of adjuvant or a mixture of adjuvants to hPTH(1-34) is between 10:1 to 0.5:1. In the most preferred embodiment the ratio of adjuvant or a mixture of adjuvants to hPTH(1-34) is between 5:1 to 0.5:1

In one embodiment of the invention, the PTH-based agent comprises in the range of approximately 1-30 wt. % of the coating formulation.

More preferably, the amount of PTH-based agent contained in the biocompatible coating on the microprojection member is in the range of 1-1000 μg, even more preferably, in the range of 10-100 μg.

Preferably, the coating formulations have a viscosity less than approximately 500 centipoise and greater than 3 centipose.

In one embodiment of the invention, the coating thickness is less than 25 microns, more preferably, less than 10 microns as measured from the microprojection surface. The desired coating thickness is dependent upon several factors, including the required dosage and, hence, coating thickness necessary to deliver the dosage, the density of the microprojections per unit area of the sheet, the viscosity and concentration of the coating composition and the coating method chosen. The thickness of coating 35 applied to microprojections 34 can also be adapted to optimize stability of the PTH-based agent. For example, Applicant's have found that as the % drug content is duced and the sucrose content increased, drug stability is enhanced.

In all cases, after a coating has been applied, the coating formulation is dried onto the microprojections 34 by various means. In a preferred embodiment of the invention, the coated microprojection member 30 is dried in ambient room conditions. However, various temperatures and humidity levels can be used to dry the coating formulation onto the microprojections. Additionally, the coated member can be heated, stored under vacuum or over desiccant, lyophilized, freeze dried or similar techniques used to remove the residual water from the coating.

It will be appreciated by one having ordinary skill in the art that in order to facilitate drug transport across the skin barrier, the present invention can also be employed in conjunction with a wide variety of iontophoresis or electrotransport systems, as the invention is not limited in any way in this regard. Illustrative electrotransport drug delivery systems are disclosed in U.S. Pat. Nos. 5,147,296, 5,080,646, 5,169,382 and 5,169,383, the disclosures of which are incorporated by reference herein in their entirety.

The term “electrotransport” refers, in general, to the passage of a beneficial agent, e.g., a drug or drug precursor, through a body surface such as skin, mucous membranes, nails, and the like. The transport of the agent is induced or enhanced by the application of an electrical potential, which results in the application of electric current, which delivers or enhances delivery of the agent, or, for “reverse” electrotransport, samples or enhances sampling of the agent. The electrotransport of the agents into or out of the human body may by attained in various manners.

One widely used electrotransport process, iontophoresis, involves the electrically induced transport of charged ions. Electroosmosis, another type of electrotransport process involved in the transdermal transport of uncharged or neutrally charged molecules (e.g., transdermal sampling of glucose), involves the movement of a solvent with the agent through a membrane under the influence of an electric field. Electroporation, still another type of electrotransport, involves the passage of an agent through pores formed by applying an electrical pulse, a high voltage pulse, to a membrane.

In many instances, more than one of the noted processes may be occurring simultaneously to different extents. Accordingly, the term “electrotransport” is given herein its broadest possible interpretation, to include the electrically induced or enhanced transport of at least one charged or uncharged agent, or mixtures thereof, regardless of the specific mechanism(s) by which the agent is actually being transported. Additionally, other transport enhancing methods, such as sonophoresis or piezoelectric devices, can be used in conjunction with the invention.

### EXAMPLES

The following examples are given to enable those skilled in the art to more clearly understand and practice the present invention. They should not be considered as limiting the scope of the invention but merely as being illustrated as representative thereof.

### Example 1

Validation reverse-phase and size-exclusion high pressure liquid chromatography (RP-HPLC and SEC-HPLC, respectively) were used to quantify PTH oxidation resulting from exposure to the sterilizing radiation. Formulations of hPTH(1-34) were prepared, comprising 20% w/w HPTH, 20% sucrose, 0.2% polysorbate 20 and 0.03% EDTA. The PTH formulations were coated onto microprojection arrays, which were then enclosed in glass vials. The coated arrays were irradiated with 7, 14 or 21 kGy radiation doses, from either gamma radiation or e-beam, under dry ice or an ambient temperature. As shown in FIG. 5, both gamma radiation and e-beam exposure decreased the purity of PTH relative to a control formulation. Tables 1 and 2 give the data corresponding to the purity and degradation products for gamma radiation and e-beam, respectively.

TABLE 1

| | Irradiation | PTH(1-34) | Oxidation | | | Total | Other | |
|---|---|---|---|---|---|---|---|---|
| | Conditions | Purity | RRT | | | Oxidation | impurities | |
| Lot No. | Gamma | (%) | 0.27 | 0.51 | 0.65 | (%) | RRT | Aggregation |
| 7822-90-1 | None | 95.7 | 0.06 | 0.26 | 1.0 | 1.4 | 0.36 | 0.10 |
| 7822-90- | 21 kGY, | 72.5 | 5.7 | 5.0 | 12.2 | 22.9 | 0.42 | 0.37 |
| 2B | ambient | | | | | | | |
| 7822-90- | 21 kGY, | 38.5 | 14.8 | 12.9 | 26.8 | 54.5 | 0.40 | 0.13 |
| 2A | dry ice | | | | | | | |
| 7822-90- | 14 kGy, | 55.0 | 9.0 | 11.5 | 21.6 | 42.1 | 0.25 | 0.21 |
| 3B | ambient | | | | | | | |
| 7822- | 14 kGy, | 64.2 | 12.5 | 5.7 | 12.8 | 31.0 | 0.40 | 0.10 |
| 980-3A | dry ice | | | | | | | |
| 7822-90- | 7 kGY, | 77.1 | 1.7 | 5.7 | 12.8 | 20.2 | 0.30 | 0.10 |
| 4B | ambient | | | | | | | |
| 7822-90- | 7 kGy, | 82.6 | 1.8 | 5.1 | 7.2 | 13.8 | 0.39 | 0.10 |
| 4A | dry ice | | | | | | | |

TABLE 2

| | Irradiation | PTH(1-34) | Oxidation | | | Total | Other | |
|---|---|---|---|---|---|---|---|---|
| | Conditions | Purity | RRT | | | Oxidation | impurities | |
| Lot No. | E-beam | (%) | 0.27 | 0.51 | 0.65 | (%) | RRT | Aggregation |
| 7822-90-1 | None | 95.7 | 0.06 | 0.26 | 1.0 | 1.4 | 0.36 | 0.10 |
| 7822-90- | 21 kGY, | 89.7 | 0.8 | 2.1 | 3.7 | 6.6 | 1.02 | 0.27 |
| 5B | ambient | | | | | | | |
| 7822-90- | 21 kGY, | 62.0 | 13.5 | 7.5 | 11.2 | 32.2 | 0.38 | 0.17 |
| 5A | dry ice | | | | | | | |
| 7822-90- | 14 kGy, | 62.8 | 5.1 | 9.2 | 19.4 | 33.7 | 0.29 | 0.20 |
| 1B | ambient | | | | | | | |
| 7822- | 14 kGy, | 75.4 | 6.1 | 5.3 | 10.2 | 21.6 | 0.22 | 0.15 |
| 980-1A | dry ice | | | | | | | |
| 7822-90- | 7 kGY, | 79.7 | 1.3 | 6.2 | 8.4 | 15.9 | 0.41 | 0.12 |
| 2B | ambient | | | | | | | |
| 7822-90- | 7 kGy, | 87.5 | 1.5 | 2.9 | 5.1 | 9.5 | 0.27 | 0.10 |
| 2A | dry ice | | | | | | | |

As can be seen from Tables 1 and 2, the largest increase in degradation products occurred at the relative retention times (RRT), which correspond to three different forms of oxidized PTH. Although the level of oxidation generally increased with radiation dose and temperature, the 21 kGY dose, particularly at an ambient temperature, experienced less degradation.

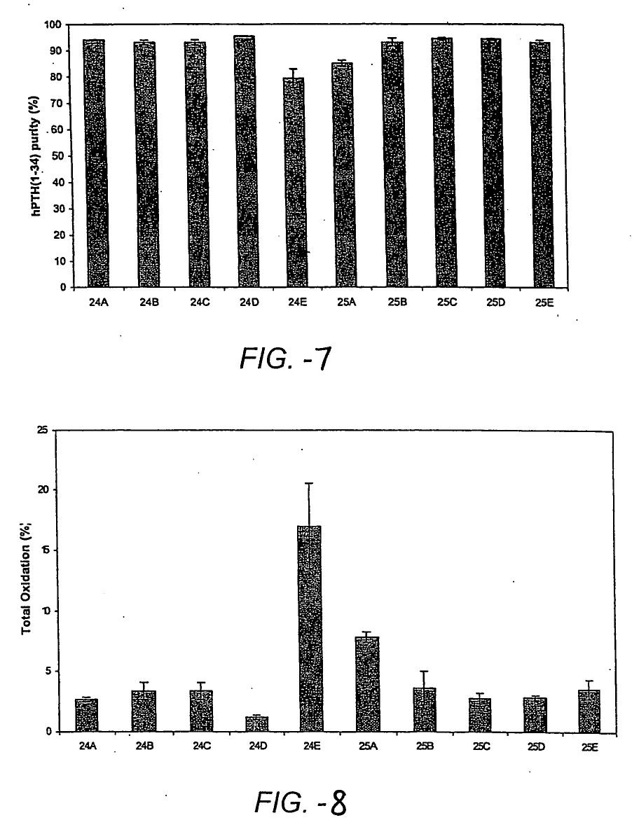

The above data also show that other degradation pathways do not play prominent roles. For example, deamidated products for the irradiated formulations were comparable to the control formulation. Further, as shown in FIG. 6, aggregation following irradiation was relatively low.

This example demonstrates that e-beam sterilization causes somewhat less degradation than gamma radiation, on the order of 5-10% less at the high radiation doses. Further, the low radiation dose of 7 kGy under dry ice experienced the least loss of purity.

### Example 2

Formulations of hPTH(1-34) were prepared as set forth in Example 1, and coated on microprojection arrays. In addition to the standard 1:1 sucrose:hPTH formulations discussed above, the effect of antioxidants was assessed by adding to selected samples 5% w/w methionine or 3.3% w/w ascorbic acid. The coated arrays were placed in nitrogen purged heat-sealed foil pouches or glass vials. Also, one of the samples was subjected to ethylene oxide sterilization as a comparison. The remaining samples were subjected to dose of gamma radiation of either 7, 14 or 21 kGy under dry ice or an ambient temperature. As discussed above, the purity and degradation of the PTH formulations was assessed using RP-HPLC and SEC-HPLC. Table 3 summarizes the irradiation protocol.

TABLE 3

| | | | | Gamma | | |
|---|---|---|---|---|---|---|
| | | | | irradiation | | |
| | | | | Dose (kGy) | | |
| Lot. No. | Conditions | Packaging | Antioxidant | 7 | 14 | 21 |
| 24A | Shipping | Pouch | No | | | |
| | Controls | | | | | |
| 24B | Dry Ice | Pouch | No | X | | |
| 24C | Dry Ice | Pouch | No | | X | |
| 24D | Shipping | Glass Vial | No | | | |
| | Controls | | | | | |
| 24E | Ambient | Glass Vial | No | | | X |
| | Temperature | | | | | |
| 25A | Ethylene Oxide | Glass Vial | No | | | |
| 25B | Dry Ice | Pouch | Methionine | | X | |
| 25C | Shipping | Pouch | Methionine | | | |
| | Controls | | | | | |
| 25D | Shipping | Pouch | Ascorbic Acid | | | |
| | Controls | | | | | |
| 25E | Dry Ice | Pouch | Ascorbic Acid | | X | |

The purity of the hPTH on the irradiated microprojection arrays is shown in FIG. 7. As can be seen, gamma irradiation did not significantly degrade hPTH, except for the 21 kGy dose delivered to the array packaged in a glass vial at ambient temperature. The other samples receiving gamma radiation retained a purity comparable to that of the controls. The sample sterilized by ethylene oxide experienced considerable degradation, probably due to the high relative humidity conditions attendant with this method of sterilization and the hygroscopic nature of hPTH. Further, the morphology of the ethylene oxide sterilized sample was altered substantially.

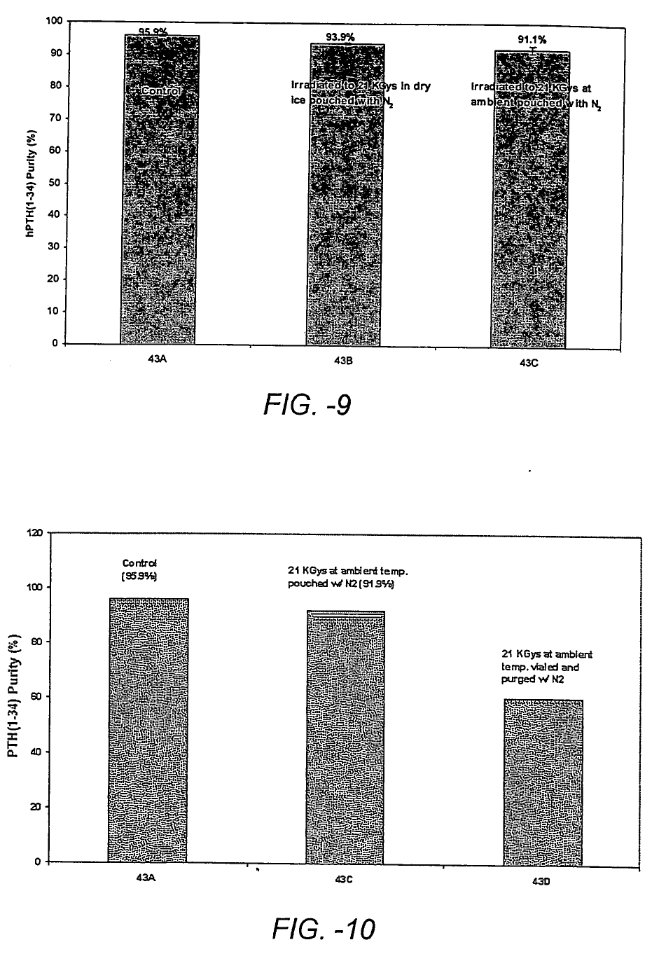

As shown in FIG. 8, the majority of the degradation can be attributed to oxidation. Moreover, degradation was severe for the sample packaged in a glass vial and irradiated at an ambient temperature and for the sample sterilized by ethylene oxide. The irradiated samples packaged in the foil pouches exhibited minimal oxidation, probably due to the inert and relatively dry atmosphere within the sealed pouch. The deamidation and aggregation degradation products were not prominent.

### Example 3

Formulations of hPTH(1-34) were prepared as set forth in Example 1, and coated on microprojection arrays. Certain arrays were assembled with a polycarbonate retainer ring and an adhesive. The arrays were sealed in foil pouches purged with nitrogen or ambient air or in glass vials. The arrays were exposed to 14 or 21 kGy of gamma radiation under dry ice or an ambient temperature. As discussed above, the purity and degradation of the PTH formulations was assessed using RP-HPLC and SEC-HPLC.

As shown in FIG. 9, the coated arrays irradiated at 21 kGy while packaged inside a nitrogen purged foil pouch did not suffer a significant loss in purity relative to the control. There was a less than 4% increase in overall oxidation at an ambient temperature and a less than 2% increase under dry ice. Thus, an hPTH(1-34) coated microprojection array is terminally sterilizable using gamma irradiation.

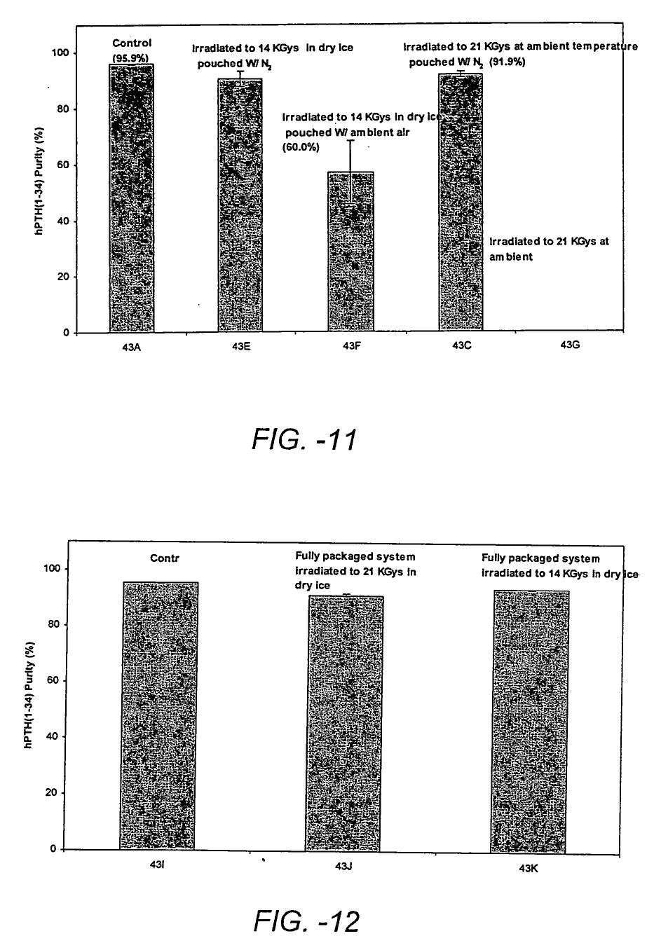

FIG. 10 illustrates the effect of packaging by comparing coated arrays irradiated at 21 kGy under an ambient temperature while packaged inside a nitrogen purged foil pouch or a nitrogen purged glass vial. Consistent with the results in FIG. 9, the sample irradiated inside the pouch experienced only an approximately 2% increase in oxidation. In contrast, the sample packaged in the glass vial suffered approximately 40% oxidation under the same irradiation conditions. This result suggests that a glass vial is not a viable barrier to prevent air/nitrogen exchange before and during irradiation.

To assess the protective aspects of the nitrogen purged foil pouch, coated arrays were irradiated at 14 kGy under dry ice and an ambient temperature, inside a foil pouch purged with nitrogen or ambient air. As shown in FIG. 11, at both temperatures, samples in the nitrogen purged pouch maintained purity comparable to the controls. In contrast, the samples sealed with ambient air suffered major oxidation at both temperatures. These results suggest that the inert gaseous medium is the key factor in protecting hPTH from degradation upon irradiation, rather than the foil pouch itself. Accordingly, minimizing oxygen and humidity in the gaseous medium surrounding the coated arrays during irradiation apparently plays a critical role in preventing degradation.

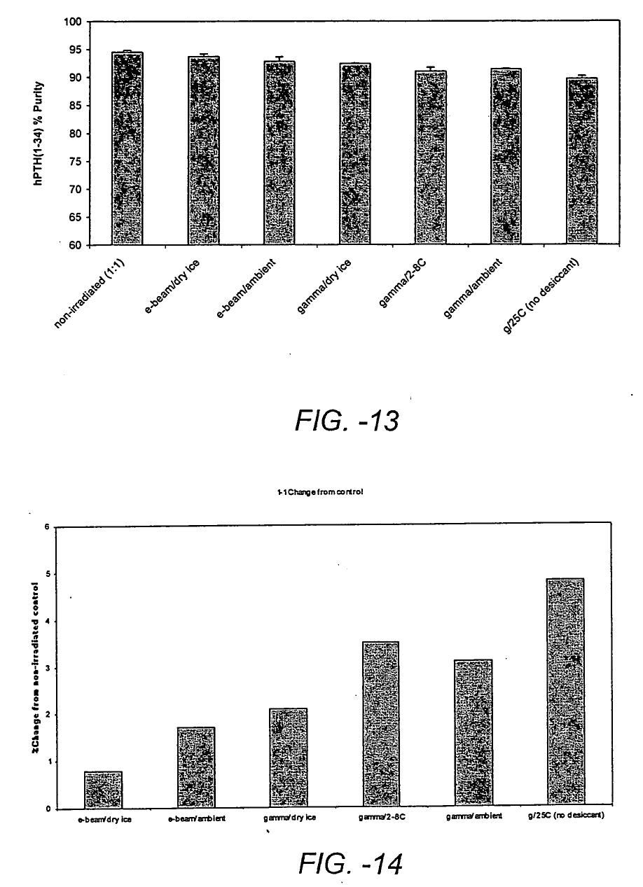

Next, coated arrays assembled with a polycarbonate retainer ring and an acrylate adhesive were packaged inside a nitrogen purged foil pouch and irradiated at 14 and 21 kGy under dry ice. As shown in FIG. 12, both samples had adequate stability following irradiation.

### Example 4

In this example, formulations of hPTH(1-34) were prepared, comprising 20% w/w HPTH, 20% sucrose or 40% sucrose, 0.2% polysorbate 20 and 0.03% EDTA. The arrays were assembled with a polycarbonate retainer ring and an adhesive. The arrays were sealed in foil pouches purged with nitrogen. Certain samples included a 4 Å molecular seive desiccant. The arrays were exposed to 15 kGy of gamma or e-beam radiation at under dry ice, 2-8° C. or an ambient temperature. As discussed above, the purity and degradation of the PTH formulations was assessed using RP-HPLC and SEC-HPLC.

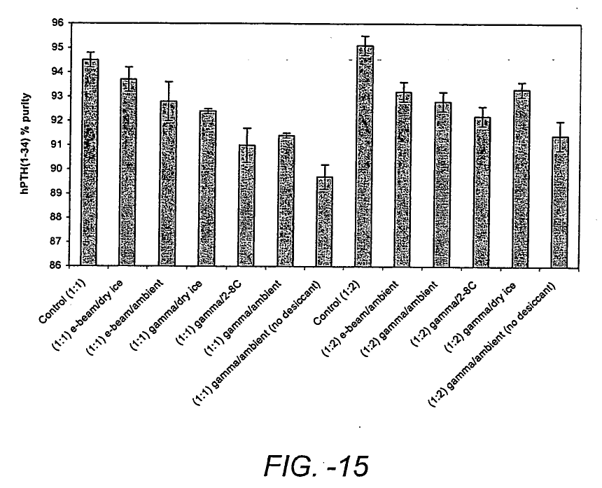

As shown in FIG. 13, e-beam sterilization causes less degradation than gamma irradiation at the same temperature. Further, the sample without a desiccant suffered the most degradation, reinforcing the importance of minimizing moisture in the environment surrounding the coated array during irradiation. The percentage change in purity for these samples is shown in FIG. 14.

FIG. 15 compares the purity of formulations of 1:1 and 2:1 sucrose:hPTH following gamma or e-beam irradiation. These results indicate that the amount of sucrose in the coating formulations did not have a significant impact on the stability of hPTH(1-34) during irradiation.

As shown by the above examples and discussion, microprojection members having a coating formulation including a PTH-based agent can be terminally sterilized by either gamma irradiation or e-beam treatment with only a minor reduction in chemical purity using the methods of the invention. Preferably, the packaging of the microprojection members is adapted to provide an inert atmosphere with relatively low humidity during the terminal sterilization process. For example, a sealed foil pouch purged with dry nitrogen and containing desiccant has a significant stabilizing effect. Also preferably, the microprojection member is mounted on a pre-dried retainer ring prior to packaging.

Further, product degradation can also be reduced during the terminal sterilization process by adjusting the temperature or by reducing the sterilization dose.

Without departing from the spirit and scope of this invention, one of ordinary skill can make various changes and modifications to the invention to adapt it to various usages and conditions. As such, these changes and modifications are properly, equitably, and intended to be, within the full range of equivalence of the following claims.

What is claimed is:

1. A method for terminally sterilizing a transdermal device adapted to deliver a PTH-based agent, comprising the steps of:
providing a microprojection member having a plurality of microprojections that are adapted to pierce the stratum comeum of a patient having a biocompatible coating disposed on said microprojection member, said coating being formed from a coating formulation having at least one PTH-based agent disposed thereon; and
exposing said microprojection member to radiation selected from the group consisting of gamma radiation and e-beam, wherein said radiation is sufficient to reach a desired sterility assurance level.

2. The method of claim 1, further comprising the step of sealing said microprojection member inside packaging adapted to control environmental conditions surrounding said microprojection member.

3. The method of claim 2, wherein said packaging comprises a foil pouch.

4. The method of claim 2, further comprising the step of sealing a desiccant inside said packaging.

5. The method of claim 2, further comprising the step of mounting said microprojection member on a pre-dried retainer ring prior to sealing said microprojection member inside said packaging.

6. The method of claim 4, further comprising the step of mounting said microprojection member on a pre-dried retainer ring prior to sealing said microprojection member inside said packaging.

7. The method of claim 2, further comprising the step of purging said packaging with an inert gas prior to sealing said microprojection member.

8. The method of claim 7, wherein said inert gas comprises nitrogen.

9. The method of claim 2, wherein said step of exposing said microprojection member to radiation occurs at approximately −78.5-25° C.

10. The method of claim 2, wherein said step of exposing said microprojection member to radiation occurs at an ambient temperature.

11. The method of claim 2, wherein said step of exposing said microprojection member to radiation comprises delivering in the range of approximately 5 to 50 kGy.

12. The method of claim 2, wherein said step of exposing said microprojection member to radiation comprises delivering approximately 7 kGy.

13. The method of claim 2, wherein said step of exposing said microprojection member to radiation comprises delivering approximately 21 kGy.

14. The method of claim 2, wherein said step of exposing said microprojection member to radiation comprises delivering radiation at a rate of greater than approximately 3.0 kGy/hr.

15. The method of claim 2, wherein said sterility assurance level is 10⁻⁶.

16. The method of claim 2, further comprising the step of adding an antioxidant to said coating formulation.

17. A method for terminally sterilizing a transdermal device adapted to deliver a PTH-based agent, comprising the steps of:
providing a microprojection member having a plurality of microprojections that are adapted to pierce the stratum comeum of a patient having a biocompatible coating disposed on said microprojection member, said coating being formed from a coating formulation having at least one PTH-based agent disposed thereon;
sealing said microprojection member with a desiccant inside packaging purged with nitrogen and adapted to control environmental conditions surrounding said microprojection member; and
exposing said microprojection member to radiation selected from the group consisting of gamma radiation and e-beam radiation, wherein said radiation is sufficient to reach a desired sterility assurance level.

18. The method of claim 17, further comprising the step of mounting said microprojection member on a pre-dried retainer ring prior to sealing said microprojection member inside said packaging.

19. The method of claim 17, wherein said step of exposing said microprojection member to radiation comprises delivering a dose of radiation in the range of approximately 7-21 kGy.

20. The method of claim 19, wherein said step of exposing said microprojection member to radiation occurs at a temperature up to approximately 25° C.

21. The method of claim 17, wherein said PTH-based agent retains at least approximately 96% of initial purity.

22. The method of claim 21, wherein said PTH-based agent retains at least approximately 98% of initial purity.

23. A method for terminally sterilizing a transdermal device adapted to deliver a PTH-based agent, comprising the steps of:
providing a microprojection member having a plurality of microprojections that are adapted to pierce the stratum corneum of a patient having a biocompatible coating disposed on said microprojection member, said coating being formed from a coating formulation having at least one PTH-based agent disposed thereon;
sealing said microprojection member inside packaging purged with an inert gas and adapted to control environmental conditions surrounding said microprojection member; and
exposing said microprojection member to e-beam radiation, wherein said radiation is sufficient to reach a desired sterility assurance level.

24. A method for terminally sterilizing a transdermal device adapted to deliver a PTH-based agent, comprising the steps of:
providing a microprojection member having a plurality of microprojections that are adapted to pierce the stratum comeum of a patient having a biocompatible coating disposed on said microprojection member, said coating being formed from a coating formulation having at least one PTH-based agent disposed thereon;
placing said microprojection member inside packaging adapted to control environmental conditions;
reducing moisture content inside said packaging;
sealing said microprojection member with said packaging; and
exposing said microprojection member to radiation selected from the group consisting of gamma radiation and e-beam, wherein said radiation is sufficient to reach a desired sterility assurance level.

25. A transdermal system, adapted to deliver a PTH-based agent, comprising:
a microprojection member including a plurality of microprojections that are adapted to pierce the stratum comeum of a patient having a biocompatible coating disposed on said microprojection member, said coating being formed from a coating formulation having at least one PTH-based agent disposed thereon; and
packaging purged with an inert gas and adapted to control environmental conditions sealed around said microprojection member;
wherein said sealed package has been exposed to radiation to sterilize the microprojection member.

26. The system of claim 25, further comprising a desiccant sealed inside said packaging with said microprojection member.

27. The system of claim 25, wherein said microprojection member is mounted on a pre-dried retainer ring.

28. The system of claim 25, wherein said packaging is purged with nitrogen.

29. The system of claim 25, wherein said packaging comprises a foil pouch.

30. The system of claim 25, wherein said PTH-based agent comprises hPTH (1-34).

31. A transdermal system, adapted to deliver a PTH-based agent, comprising:
a microprojection member including a plurality of microprojections that are adapted to pierce the stratum comeum of a patient;
a hydrogel formulation having at least one PTH-based agent, wherein said hydrogel formulation is in communication with said microprojection member; and
packaging purged with an inert gas and adapted to control environmental conditions sealed around said microprojection member;
wherein said sealed package has been exposed to radiation to sterilize the microprojection member.

32. A transdermal system, adapted to deliver a PTH-based agent, comprising:
a microprojection member including a plurality of microprojections that are adapted to pierce the stratum comeum of a patient;
a solid film disposed proximate said microprojection member, wherein said solid film is made by casting a liquid formulation comprising at least one PTH-based agent, a polymeric material, a plasticizing agent, a surfactant and a volatile solvent; and
packaging purged with an inert gas and adapted to control environmental conditions sealed around said microprojection member;
wherein said sealed package has been exposed to radiation to sterilize the microprojection member.
